(19) United States
(12) Patent Application Publication
(10) Pub. No.: US 2023/0373784 A1
(43) Pub. Date: Nov. 23, 2023

(54) METHOD AND APPARATUS FOR PRODUCING PRODUCT GAS AND USE

(71) Applicant: TEKNOLOGIAN TUTKIMUSKESKUS VTT OY, Espoo (FI)

(72) Inventors: Pekka SIMELL, Espoo (FI); Ilkka HANNULA, Espoo (FI)

(21) Appl. No.: 18/247,217

(22) PCT Filed: Sep. 29, 2021
(86) PCT No.: PCT/FI2021/050643

§ 371 Date: Mar. 29, 2023

(30) Foreign Application Priority Data
Oct. 1, 2020 (FI) 20205961

(51) Int. Cl. C01B 3/40; B01J 12/00; B01J 19/00; C07C 1/04

(52) U.S. Cl. CPC C01B 3/40; B01J 12/007; B01J 19/0013; C07C 1/0485; C01B 2203/0238; C01B 2203/0261; C01B 2203/1064; C01B 2203/1082; C01B 2203/1023; C01B 2203/062; C01B 2203/061; C01B 2203/065; C01B 2203/085; B01J 2219/00135

(57) ABSTRACT

The invention relates to a method and apparatus for producing a product gas from a feed comprising at least carbon dioxide, hydrogen and hydrocarbons. The feed is supplied to a reactor comprising a catalyst, the catalyst is heated electrically, the feed is supplied through the catalyst and a reaction is performed at least between carbon dioxide (CO₂) and hydrogen (H₂) in the presence of the catalyst in the reactor, and the product gas comprising at least carbon monoxide (CO) and hydrogen (H₂) is formed in the reactor. Further, the invention relates to the use of the method.

### FIELD

The application relates to a method defined in claim 1 and an apparatus defined in claim 12 for producing a product gas from a feed comprising at least carbon dioxide, hydrogen and hydrocarbons. Further, the application relates to a use of the method defined in claim 17.

### BACKGROUND

Known from the prior art is to produce hydrocarbons by a Fischer-Tropsch synthesis. The Fischer-Tropsch synthesis requires a mixture of H₂ and CO as feed.

Further, it is known from the prior art that carbon dioxide may be converted to carbon monoxide by RWGS (reverse water gas shift) reaction. A soot formation is problem in RWGS reactors.

### OBJECTIVE

The objective is to solve the above problems. The objective is to disclose a new type of method and apparatus for producing carbon monoxide from carbon dioxide effectively. Further, the objective is to disclose a new type of method and apparatus for treating streams comprising carbon dioxide and hydrocarbons. Further, the objective is to improve F-T process, RWGS-process and/or other refining processes. Further, the objective is to disclose the method and apparatus, in which the process can be heated electrically. Further, the objective is to increase the achievable conversion of power to fuels and chemicals.

### SUMMARY

The method and apparatus and use are characterized by what are presented in the claims.

In the method and apparatus, a product gas is produced from a feed comprising at least carbon dioxide, hydrogen and hydrocarbons, in a reactor in the presence of a catalyst.

### BRIEF DESCRIPTION OF THE DRAWINGS

The accompanying drawings, which are included to provide further understanding of the invention and constitute a part of this specification, illustrate some embodiments of the invention and together with the description help to explain the principles of the invention. In the drawings:

FIG. 1 is a flow chart illustration of a process according to one embodiment, and

FIG. 2 is a flow chart illustration of a process according to another embodiment.

### DETAILED DESCRIPTION

In a method for producing a product gas from a feed comprising at least carbon dioxide, hydrogen and hydrocarbons, the feed (1) is supplied to a reactor (2) comprising a catalyst, the catalyst is heated electrically, the feed is supplied through the catalyst and a reaction is performed at least between carbon dioxide (CO₂) and hydrogen (H₂) in the presence of the catalyst in the reactor, and the product gas (3) comprising at least carbon monoxide (CO) and hydrogen (H₂) is formed in the reactor. Preferably carbon dioxide is converted to carbon monoxide during the reaction in the reactor (2).

An apparatus for producing a product gas from a feed comprising at least carbon dioxide, hydrogen and hydrocarbons, comprises a reactor (2) comprising a catalyst, at least one heating device for heating the catalyst electrically, at least one feeding device for feeding the feed (1) to the reactor (2) in which the feed is supplied through the catalyst and in which a reaction at least between carbon dioxide and hydrogen is performed in the presence of the catalyst and the product gas (3) comprising at least carbon monoxide and hydrogen is formed.

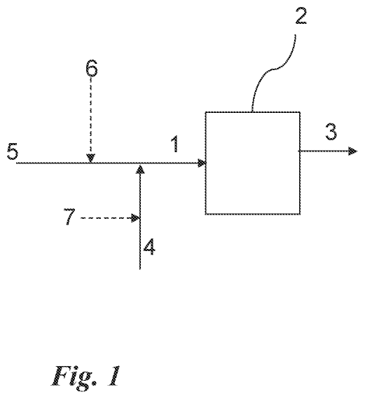

One embodiment of the method and the apparatus is shown in FIG. 1.

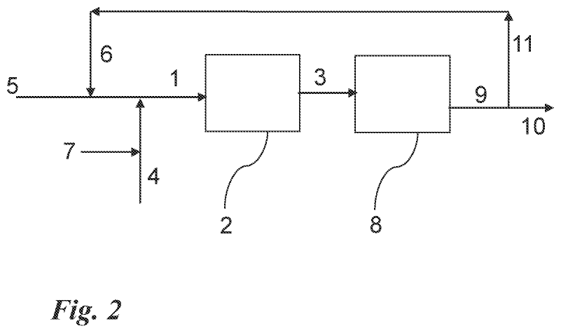

Another embodiment of the method and the apparatus is shown in FIG. 2.

The feed comprises hydrogen. The hydrogen may be fed with the feed. In one embodiment, hydrogen (5) is fed to the reactor.

In one embodiment, oxygen (7) is fed to the reactor. The oxygen may be fed with the feed or as a separate oxygen feed to the reactor. When the oxygen is supplied to the reactor, a partial oxidation can be carried out in the reactor. In one embodiment, an amount of the oxygen which is supplied to the reactor is based on process conditions and/or a desired product distribution. Controlling catalyst coking can be performed using small amount of oxygen in the feed.

In one embodiment, the feed (1) is in gaseous form. In one embodiment, the feed comprises oxygen. In one embodiment, the feed comprises at least carbon dioxide, oxygen, hydrogen and hydrocarbons. The feed may contain also other compounds. In this context, the feed means any feed into the reactor (2) in which carbon dioxide is converted to carbon monoxide. The feed can be supplied through the catalyst in the reactor. In one embodiment, the feed is formed from different components before the supply into the reactor.

In one embodiment, oxygen (7) is added to a carbon dioxide stream (4), the carbon dioxide stream (4) is combined with a hydrogen based stream (5) to form the feed (1) and a hydrocarbon containing stream (6) is supplied to the hydrogen based stream (5) before combining with the carbon dioxide stream (4). In one embodiment, oxygen (7) and a carbon dioxide stream (4) are added to a hydrogen based stream (5) to form the feed (1) and a hydrocarbon containing stream (6) is supplied to the hydrogen based stream (5) before the addition of the carbon dioxide stream (4) and oxygen. In one embodiment, oxygen content is below 5 vol-% in the feed.

Preferably, the feed (1) comprises carbon dioxide, e.g. carbon dioxide stream (4). In this context, the carbon dioxide stream (4) means any carbon dioxide stream or carbon dioxide based stream. In one embodiment, the carbon dioxide stream contains at least carbon dioxide, and it may contain also a little amount of hydrocarbons.

In this context, the hydrocarbons mean any hydrocarbons. In one embodiment, the hydrocarbons are at least partly recycled hydrocarbons. In this context, the hydrocarbon containing stream (6) means any stream which comprises at least hydrocarbons. In one embodiment, the hydrocarbons or the hydrocarbon containing stream (6) comprises light hydrocarbons, preferably C1-C6 hydrocarbons. In one embodiment, the hydrocarbons or the hydrocarbon containing stream (6) comprises hydrocarbons which are C1-C30 hydrocarbons. In one embodiment, the hydrocarbon containing stream (6) comprises hydrocarbons and, further, hydrogen, carbon monoxide and/or carbon dioxide. In one embodiment, the hydrocarbons or the hydrocarbon containing stream is added to the hydrogen based stream (5), the carbon dioxide stream (4) or the feed (1). In one embodiment, the carbon dioxide stream (4) comprises the hydrocarbons.

In this context, the hydrogen based stream (5) means any stream which comprises hydrogen. Preferably, the hydrogen based stream comprises mainly hydrogen, i.e. it mainly consists of hydrogen.

In one embodiment, the apparatus comprises at least one feeding device for supplying the oxygen (7) to the carbon dioxide stream (4), the carbon dioxide stream (4) to the hydrogen based stream (5) and/or the hydrocarbon containing stream (6) to the hydrogen based stream (5). In one embodiment, the apparatus comprises at least one feeding device for feeding the oxygen, the carbon dioxide stream, the hydrogen based stream and/or the hydrocarbon containing stream. In this context, the feeding device may be any device by which a desired stream or feed can be fed or supplied to the apparatus, to the reactor or to their parts, e.g. any feeder, feeding device, compressor, pump, pipe, feed inlet or other suitable feeding equipment or their combinations. In one embodiment, the apparatus comprises the feeding device for feeding oxygen, and said feeding device is arranged to feed the oxygen or to stop the oxygen feed during the process.

In one embodiment, a partial oxidation is carried out in the reactor (2). Preferably, the partial oxidation is an exothermic reaction. Then the feed is treated by means of the partial oxidation in the reactor (2) so that carbon dioxide reacts with hydrogen in the reactor in presence of oxygen and some heat is formed during the reaction. Preferably, also the carbon monoxide is formed from carbon dioxide in the reactor. In one embodiment, also a reverse water gas shift (RWGS) type reaction is carried out in the reactor in order to convert carbon dioxide to carbon monoxide. The reverse water gas shift (RWGS) reaction is an endothermic reaction. Preferably, the partial oxidation reaction brings some heat for the reaction in addition to electrical heating, where carbon dioxide is converted to carbon monoxide. Preferably, the reactions in the reactor are based on the combination of the partial oxidation reaction and the reaction for converting carbon dioxide to carbon monoxide. In one embodiment, the reactor is based on a combined CPDX and RWGS reactor.

In one embodiment, the reactor (2) is a tube reactor or tubular reactor. In one embodiment, the reactor is a partial oxidation reactor in which the partial oxidation is carried out. In one embodiment, the reactor is a catalytic partial oxidation (CPDX) reactor. In one embodiment, the reactor is a CPDX reactor in which RWGS reaction (reverse water gas shift reaction) is also carried out. In one embodiment, hydrogen rich syngas is formed in the reactor, such as in the CPDX reactor. In one embodiment, carbon monoxide rich gas is formed in the reactor, such as in the RWGS reactor.

In this context, the catalyst may be any catalyst, catalyst structure or catalyst bed, which comprises at least a catalytic material.

In one embodiment, the catalyst is a catalyst bed comprising at least a catalytic material. In one embodiment, the catalyst is a catalyst structure which comprises the catalytic material, e.g. on the surface of the structure.

In one embodiment, the catalyst is a porous material structure with a catalytic material. Then the reactor (2) comprises the porous material structure which comprises the catalytic material. Preferably, the porous material structure is formed from porous material. In one embodiment, the porous material structure consists of porous material which comprises the catalytic material. In one embodiment, the porous material structure comprises an inner part which is formed at least in part from the porous material comprising the catalytic material and in which at least one reactant is arranged to flow into the inner part and after that through the porous material to form a product, and a shell structure which surrounds the inner part and a space between the inner part and the shell structure in which the product formed from the reactant or reactants in the porous material is arranged to flow out from the reactor. In one embodiment, the product is rinsed from the surface of the inner part, e.g. by means of a scavenging agent, and is arranged to flow out from the porous material structure via the space between the inner part and the shell structure.

In one embodiment, the porous material of the porous material structure comprises porous metallic, ceramic and/or composite material. The porous material contains pores. In one embodiment, the porous material is catalytically coated. In one embodiment, the catalytic material is arranged on a surface of the porous material. In one embodiment, the catalytic material is arranged on surfaces of the pores of the porous material in the porous material structure. In one embodiment, the porous material is produced from start materials comprising the catalytic material. In one embodiment, the catalytic material is added by coating onto the porous material. The porous material may be formed such that desired pore structure, controlled porosity and/or high specific surface area can be provided to the porous material. In one embodiment, the porous material comprises pores with size of below 500 μm, in one embodiment below 150 μm, and in one embodiment below 100 inn.

In one embodiment, the porous material is produced by coating an organic space holder material with at least one catalytic material or catalytic material of the catalyst to form a coated organic space holder material, by mixing the coated organic space holder material with a carrier material to form a mixture, and by removing the organic space holder material and sintering the mixture to form the porous material with the catalytic material, and the porous material comprises pores. In one embodiment, the diameter of the pores is below 500 inn, in one embodiment below 150 inn, and in one embodiment below 100 inn. In one embodiment, the carrier material is selected from metal, ceramic material, alloy or their combinations, e.g. FeCrAl-alloy.

Preferably, the catalyst comprises at least the catalytic material. The catalyst or catalytic material may be formed from one or more catalytic material component. In one embodiment, the catalyst or catalytic material comprises at least metal, ceramic material, composite material and/or their combination. In one embodiment, the catalyst or catalytic material comprises metal selected from the group consisting of Ni, Co, Fe, other suitable metal, their compounds or their combinations. In one embodiment, the catalyst or catalytic material comprises metal of the noble metal group, e.g. Rh, Pd or Pt. In one embodiment, the catalyst or catalytic material is Rh/Al₂O₃ catalyst. In one embodiment, the catalyst or catalytic material is NiRh/Al₂O₃ catalyst. In one embodiment, the catalyst or catalytic material is Ni/Al₂O₃ catalyst. In one embodiment, the catalyst or catalytic material is selected from Rh/Al₂O₃ catalyst, NiRh/Al₂O₃ catalyst and Ni/Al₂O₃ catalyst. Alternatively, other suitable catalyst can be used as the catalyst or catalytic material.

Preferably, the catalyst or porous material structure is heated electrically. In one embodiment, only the catalyst or porous material structure is heated electrically. In one embodiment, the catalyst and/or porous material structure is heated resistively or inductively, e.g. using an electric resistance heating or using an induction heating. In one embodiment, the heating device is arranged to heat the catalyst and/or porous material structure by using an electric resistance heating. In one embodiment, the heating device is arranged to heat the catalyst and/or porous material structure by using an induction heating. In one embodiment, a partial oxidation is performed in the reactor for providing additional heat to the reaction.

In one embodiment, the treatment temperature is 700-1500° C. in the reactor (2). In one embodiment, the treatment temperature is preferably over 800° C. In one embodiment, the treatment temperature is 700-1000° C., and in one embodiment 800-950° C. In one embodiment, the heat is formed during the partial oxidation reaction in the reactor (2). In one embodiment, the reaction is at least started by electrically heating the catalyst. In one embodiment, the catalyst may be electrically heated during the process.

In one embodiment, pressure in the reactor (2) is 15-30 bar, and in one embodiment 17-25 bar. In one embodiment, the pressure is preferably about 20 bar. In one embodiment, the pressures are same, e.g. 15-25 bar, in the reactor and in a continuation process, e.g. in a Fischer-Tropsch process.

In one embodiment, the feed comprises hydrocarbons which are recycled hydrocarbons, e.g. an off-gas stream from a predetermined process. In one embodiment, the hydrocarbon containing stream (6) consists of recycled hydrocarbons. In one embodiment, the hydrocarbon containing stream comprises at least an off-gas stream, e.g. an off-gas stream (11) from a Fischer-Tropsch process (F-T process). In this context, the off-gas stream means any off-gas or tail gas or other undesired gas. Preferably, the off-gas stream comprises undesired components, such as light hydrocarbons, unreacted feed components, non-condensable components or their combinations. Further, the off-gas stream can comprise water. In one embodiment, the off-gas stream comprises at least light hydrocarbons, preferably C1-C6 hydrocarbons. In one embodiment, the off-gas stream comprises hydrocarbons and, further, hydrogen, carbon monoxide and/or carbon dioxide. In one embodiment, the off-gas stream from the Fischer-Tropsch process is recirculated as hydrocarbons and is added to the feed. Then the off-gas stream of the Fischer-Tropsch process can be reformed. In one embodiment, the apparatus comprises at least one recirculation device for recirculating the off-gas stream from the Fischer-Tropsch process and for adding the off-gas stream as hydrocarbons to the feed. In one embodiment, an amount of the off-gas is below 20 vol-%, in one embodiment below 10 vol-%, in the feed.

In one embodiment, the apparatus comprises at least one recovering device for recovering the product gas (3) from the reactor (2).

In this context, the product gas (3) means any product from the reactor (2). The product gas comprises one or more product components, e.g. carbon monoxide, hydrogen and/or other components. Preferably the product gas contains at least carbon monoxide and hydrogen. In one embodiment, the product gas may contain also water. The product gas may contain also other components. In one embodiment, the product gas can be post-treated after the reactor (2). In one embodiment, the product gas can be supplied to a desired treatment process, e.g. to a Fischer-Tropsch process. In one embodiment, the product gas is a syngas which can be supplied to the Fischer-Tropsch (FT) process. In one embodiment, water may be removed from the product gas after the reactor (2). In one embodiment, the product distribution of the product gas (3) may be adjusted by means of the components in the feed (1) and amounts of said components.

In one embodiment, the product gas (3) is cooled after the reactor (2). In one embodiment, the product gas is cooled to temperature of 4-300° C., and in one embodiment to about 250° C.

In one embodiment, the product gas (3) is used as a feed to a synthesis process, such as to a Fischer-Tropsch (FT) process, or a methanation, or a production of methanol, or to another suitable process.

In one embodiment, the apparatus belongs to a process arrangement in which the process arrangement comprises at least one additional device or process apparatus. In one embodiment, the process arrangement comprises a RWGS-reactor, partial oxidation reactor or their combination as the apparatus defined in this description. In one embodiment, the process arrangement comprises the RWGS-reactor as the additional device. In one embodiment, the process arrangement comprises the partial oxidation reactor as the additional device. In one embodiment, the process arrangement comprises a Fischer-Tropsh-reactor, wherein CO and H₂ are supplied from the apparatus to the Fischer-Tropsh-reactor and wherein the off-gases from the Fischer-Tropsh-reactor may be supplied to the apparatus. Any suitable Fischer-Tropsch (FT) reactor known per se can be used as the Fischer-Tropsch reactor in the process arrangement. Any suitable RWGS-reactor known per se can be used as the RWGS-reactor in the process arrangement. Any suitable the partial oxidation reactor or the catalytic partial oxidation (CPDX) reactor known per se can be used as the partial oxidation reactor in the process arrangement.

Preferably, the Fischer-Tropsch (FT) reaction is an exothermic reaction in which carbon monoxide reacts with hydrogen. In one embodiment, paraffin-rich hydrocarbons which can be considered as heavy hydrocarbons are formed from the carbon monoxide and hydrogen in the Fischer-Tropsch (FT) reaction. In one embodiment, the Fischer-Tropsch reaction is carried out by means of Co-based catalyst or Fe-based catalyst in the Fischer-Tropsch (FT) reactor (8). Alternatively, the FT reaction can be made with other suitable catalyst. In one embodiment, the Fischer-Tropsch reaction is carried out at temperature which is 150-350° C., in one embodiment 200-300° C. In one embodiment, pressure is 15-25 bar, in one embodiment about 20 bar, during the FT reaction. In one embodiment, the Fischer-Tropsch reaction takes place at around 20 bar pressure and around 200-300° C. In one embodiment, the product (9) of the Fischer-Tropsch (FT) process is a mixture of hydrocarbons. In one embodiment, the product of the FT comprises at least hydrocarbons, e.g. C5-C60 hydrocarbons, such as oil and wax components. Further, the product of the FT may comprise undesired components, such as water, light hydrocarbons, unreacted feed components and/or non-condensable components or their combinations. In one embodiment, the non-condensable components are discharged as an off-gas stream (11) from the FT product and desired fractions (10) are recovered.

In one embodiment, the off-gas stream (11) is recycled and is used as the hydrocarbon containing stream (6) in the feed (1) of the reactor (2), such as catalytic partial oxidation reactor (CPDX), and the product gas (3) from the reactor (2) is supplied to the FT reactor (8). Then the off-gases of the FT reactor can be recirculated to the reactor (2) in which the off-gases can be processed to syngas, such as carbon monoxide. In one embodiment, the reactor (2), e.g. CPDX reactor, is operated at the same pressure as the FT reactor (8) wherein the off-gas recirculation can be utilized better.

In one embodiment, the method and apparatus are based on a continuous process.

In one embodiment, the apparatus and the method is used and utilized in a production of hydrocarbons, Fischer-Tropsch (FT) process, treatment of carbon dioxide, carbon dioxide capture process, catalytic partial oxidation (CPDX) process, reforming off-gases, cracking process of naphtha and other hydrocarbon feedstocks, methanation process, production of methanol, or their combinations.

Thanks to the invention, carbon dioxide based feeds can be treated and converted easily and effectively. Thanks to the structure of the reactor can be heated effectively by means of ane electrical heating. Also, by means of the partial oxidation can be brought the necessary heat for the reaction in which carbon dioxide is converted to carbon monoxide. When the oxygen is fed to carbon dioxide stream, the reaction of the oxygen and the burning can be prevented such that the reactions do not take place too quickly, but the reactions take place with the catalyst in the reactor. By means of the invention carbon dioxide can be used as a feed for a FT process. Further, undesired products or streams comprising hydrocarbons, such as off-gases from FT processes, can be recirculated and used in the feed. By means of said recirculation the yield of oils and waxes may be improved in the FT process. Further, the yield of synthesis products from power can be increased compared to conventional solutions. Further, the invention helps controlling the carbon or coke formation.

The method and apparatus offer a possibility to treat carbon dioxide and carbon monoxide easily, and energy- and cost-effectively. The present invention provides an industrially applicable, simple and affordable way to produce carbon monoxide, and further to produce desired hydrocarbons by means of the FT reaction. The method and apparatus are easy and simple to realize in connection with production processes.

### EXAMPLES

FIG. 1 presents the method and the apparatus for producing carbon monoxide (CO) and hydrogen (H₂) from the feed comprising at least carbon dioxide (CO₂), hydrocarbons and hydrogen. A product gas (3) comprising the carbon monoxide and hydrogen are formed from a gaseous feed (1) which comprises at least carbon dioxide, hydrocarbons and hydrogen in a reactor (2). Oxygen (7) may be added to a carbon dioxide stream (4). The carbon dioxide stream (4) is combined with a hydrogen based stream (5) to form the feed (1). The feed (1) may comprise hydrocarbons or a hydrocarbon containing stream (6) is supplied to the hydrogen based stream (5) before combining with the carbon dioxide stream (4). The gaseous feed (1) is fed into the reactor (2) which comprises a porous material structure with a catalytic material. The porous material structure comprises a metal based porous material which comprises pores. The surfaces of the pores in the porous material has been coated by the catalytic material. Rh/Al₂O₃ catalyst may be used as the catalytic material in this example. The porous material structure is heated electrically by an electrical heating device. The gaseous feed is supplied through the porous material structure in the reactor.

FIG. 2 presents the method and the apparatus for producing carbon monoxide (CO) and hydrogen (H₂) from the feed comprising at least carbon dioxide (CO₂), hydrocarbons and hydrogen.

A product gas (3) comprising the carbon monoxide and hydrogen are formed from a gaseous feed (1) which comprises at least carbon dioxide, hydrocarbons, hydrogen and oxygen in a partial oxidation reactor (2). Oxygen (7) is added to a carbon dioxide stream (4). The carbon dioxide stream (4) is combined with a hydrogen based stream (5) to form the feed (1). An off-gas stream as a hydrocarbon containing stream (6) is supplied to the hydrogen based stream (5) before combining with the carbon dioxide stream (4). Then the gaseous feed (1) is fed into the reactor (2) which comprises the porous material structure with the catalytic material. The porous material structure comprises a metal based porous material which comprises pores. The surfaces of the pores in the porous material has been coated by the catalytic material. Rh/Al₂O₃ catalyst may be used as the catalytic material in this example. Alternatively, other suitable catalyst may be used. The porous material structure is heated electrically by an electrical heating device. The gaseous feed is supplied through the catalyst, such as the porous material structure, in the reactor.

The gaseous feed (1) is treated by means of a catalytical partial oxidation reaction in the reactor (2) so that the carbon dioxide reacts with hydrogen in the reactor in presence of the oxygen and heat is formed during the reaction. Simultaneously carbon dioxide is converted to carbon monoxide in the reactor. Temperature is preferably 800-950° C. and pressure is about 20 bar in the reactor (2). The product gas (3) comprising at least carbon monoxide and hydrogen is discharged from the reactor (2) and is recovered.

The product gas (3) is supplied as the feed to a Fischer-Tropsch (FT) reactor (8). The product gas may be cooled and water may be removed from the product gas before the FT reactor. The temperature is 200-300° C. after the cooling. The Fischer-Tropsch (FT) reaction is an exothermic reaction in which carbon monoxide reacts with hydrogen and paraffin-rich hydrocarbons can be formed. Temperature is preferably 200-300° C. and pressure is about 20 bar in the FT reactor.

A product (9) of the FT reactor (8) is a mixture of hydrocarbons comprising C5-C60 hydrocarbons. The product of FT comprises desired components, such as oil and wax components, and undesired components, such as light hydrocarbons, unreacted feed components and/or non-condensable components or their combinations. The desired components are recovered as product fractions (10). An off-gas stream (11) comprising undesired components from the FT reactor (8) is recycled and is used as the hydrocarbon containing stream (6) in the feed (1) of the partial oxidation reactor (2). The off-gas stream (11) comprises at least hydrocarbons, and it may comprise at least light hydrocarbons, preferably C1-C6 hydrocarbons.

Preferably, the pressure in the partial oxidation reactor (2) is same than the pressure in the FT reactor (8). Then the off-gases of the FT reactor can be recirculated to the partial oxidation reactor in which the off-gases can be processed to carbon monoxide.

The devices and equipments of the process used in these examples are known per se in the art, and therefore they are not described in any more detail in this context.

The method and apparatus are suitable in different embodiments for producing different product gases from different kinds of feeds.

The invention is not limited merely to the examples referred to above; instead many variations are possible within the scope of the inventive idea defined by the claims.

1. A method for producing a product gas from a feed comprising at least carbon dioxide, hydrogen and hydrocarbons, characterized in that the method comprises
supplying the feed (1) to a reactor (2) comprising a catalyst,
heating the catalyst electrically,
supplying the feed through the catalyst and performing a reaction at least between carbon dioxide (CO₂) and hydrogen (H₂) in the presence of the catalyst in the reactor, and
forming the product gas (3) comprising at least carbon monoxide (CO) and hydrogen (H₂) in the reactor.

2. The method according to claim 1, characterized in that hydrogen is fed to the reactor.

3. The method according to claim 1 or 2, characterized in that oxygen is fed to the reactor.

4. The method according to any one of claims 1 to 3, characterized in that the feed (1) comprises at least carbon dioxide, hydrogen, oxygen and hydrocarbons.

5. The method according to any one of claims 1 to 4, characterized in that oxygen (7) is added to a carbon dioxide stream (4), the carbon dioxide stream (4) is combined with a hydrogen based stream (5) to form the feed (1) and a hydrocarbon containing stream (6) is supplied to the hydrogen based stream (5) before combining with the carbon dioxide stream (4).

6. The method according to any one of claims 1 to 5, characterized in that a partial oxidation is carried out in the reactor (2).

7. The method according to any one of claims 1 to 6, characterized in that the catalyst is a porous material structure with a catalytic material.

8. The method according to claim 7, characterized in that the catalytic material is arranged on surfaces of pores of porous material in the porous material structure.

9. The method according to any one of claims 1 to 8, characterized in that the catalyst is Rh/Al₂O₃ catalyst.

10. The method according to any one of claims 1 to 9, characterized in that the catalyst is heated resistively or inductively.

11. The method according to any one of claims 1 to 10, characterized in that an off-gas stream from a Fischer-Tropsch process is recirculated as hydrocarbons and is added to the feed.

12. An apparatus for producing a product gas from a feed comprising at least carbon dioxide, hydrogen and hydrocarbons, characterized in that the apparatus comprises
a reactor (2) comprising a catalyst,
at least one heating device for heating the catalyst electrically, and
at least one feeding device for feeding the feed (1) to the reactor (2) in which the feed is supplied through the catalyst and in which a reaction at least between carbon dioxide and hydrogen is performed in the presence of the catalyst and the product gas (3) comprising at least carbon monoxide and hydrogen is formed.

13. The apparatus according to claim 12, characterized in that the heating device is arranged to heat the catalyst by using an electric resistance heating.

14. The apparatus according to claim 12 or 13, characterized in that the heating device is arranged to heat the catalyst by using an induction heating.

15. The apparatus according to any one of claims 12 to 14, characterized in that the apparatus comprises at least one recirculation device for recirculating an off-gas stream from a Fischer-Tropsch process as hydrocarbons to the feed.

16. The apparatus according to any one of claims 12 to 15, characterized in that the apparatus comprises a feeding device for feeding oxygen.

17. A use of the method according to any one of claims 1 to 11, characterized in that the method is used in a production of hydrocarbons, Fischer-Tropsch (FT) process, treatment of carbon dioxide, carbon dioxide capture process, catalytic partial oxidation (CPDX) process, reforming off-gases, cracking process of naphtha and other hydrocarbon feedstocks, methanation process, production of methanol, or their combinations.
